(19) United States
(12) Patent Application Publication
(10) Pub. No.: US 2009/0171163 A1
(43) Pub. Date: Jul. 2, 2009

(54) MODULAR MEDICAL DEVICES

(76) Inventors: John W. Mates, Portland, OR (US); Jeff C. Morriss, Cornelius, OR (US)

Correspondence Address: SCHWEGMAN, LUNDBERG & WOESSNER, P.A., P.O. BOX 2938, MINNEAPOLIS, MN, 55402, US

(21) Appl. No.: 11/967,773

(22) Filed: Dec. 31, 2007

(51) Int. Cl. A61B 5/00

(52) U.S. Cl. 600/300

(57) ABSTRACT

Apparatus and method provide for forming a modular medical device by selecting a combination of medical device modules and combining them to form the modular medical device, for example by plugging them together or into a common bus. In one embodiment, the modules are selected from a group of modules adapted to be used in combination to perform one or more medical functions for a particular patient. In one embodiment at least one module is adapted to store data, at least one module is adapted to perform a communication function, at least one module is used to control the medical device, at least one module is adapted to provide a sense function, and at least one module provides energy to the medical device. In another embodiment the modular medical devices may be rented or sold to patients, or prescribed by physicians and covered by medical insurance.

### TECHNICAL FIELD

The disclosed subject matter relates generally to medical technology and health delivery systems, and more particularly to modular medical devices.

### BACKGROUND

Throughout the developed world, medical technology is ubiquitous in hospitals and clinics, and increasingly commonly implanted in patients or used by patients at home. Further, use of medical technology has grown dramatically over the last quarter century to save, extend and improve the quality of life for millions of patients.

### BRIEF DESCRIPTION OF THE DRAWINGS

FIG. 1 illustrates an example construction of a medical device module according to the inventive subject matter.

FIG. 2 illustrates an example embodiment of connecting modular medical devices according to the inventive subject matter.

FIG. 3 illustrates a group or selection of modules according to the inventive subject matter.

FIGS. 4A, 4B and 4C illustrate example embodiments of modular medical devices according to the inventive subject matter.

FIG. 5A, 5B, 5C, 5D, 5E and 5F illustrate example embodiments of modular medical devices according to the inventive subject matter.

### DETAILED DESCRIPTION

In the following detailed description of example embodiments of the invention, reference is made to specific example embodiments of the invention by way of drawings and illustrations. These examples are described in sufficient detail to enable those skilled in the art to practice the invention, and serve to illustrate how the invention may be applied to various purposes or embodiments. Other embodiments of the invention exist and are within the scope of the invention, and logical, mechanical, electrical, and other changes may be made without departing from the subject or scope of the present invention. Features or limitations of various embodiments of the invention described herein, however essential to the example embodiments in which they are incorporated, do not limit other embodiments of the invention or the invention as a whole, and any reference to the invention, its elements, operation, and application do not limit the invention as a whole but serve only to define these example embodiments. The following detailed description does not, therefore, limit the scope of the invention, which is defined only by the appended claims.

According to one example embodiment, there is disclosed herein a modular medical device and a method of prescribing, distributing, and assembling such devices. Modular devices as described herein may be used for a variety of medical purposes, such as patient monitoring. Monitored parameters may include, for example, blood pressure, heart rate, blood sugar, or movement. A modular device may also, according to one example embodiment, record and/or relay readings of such parameters to medical care givers through a communication link, such as a wireless or hard wired connection.

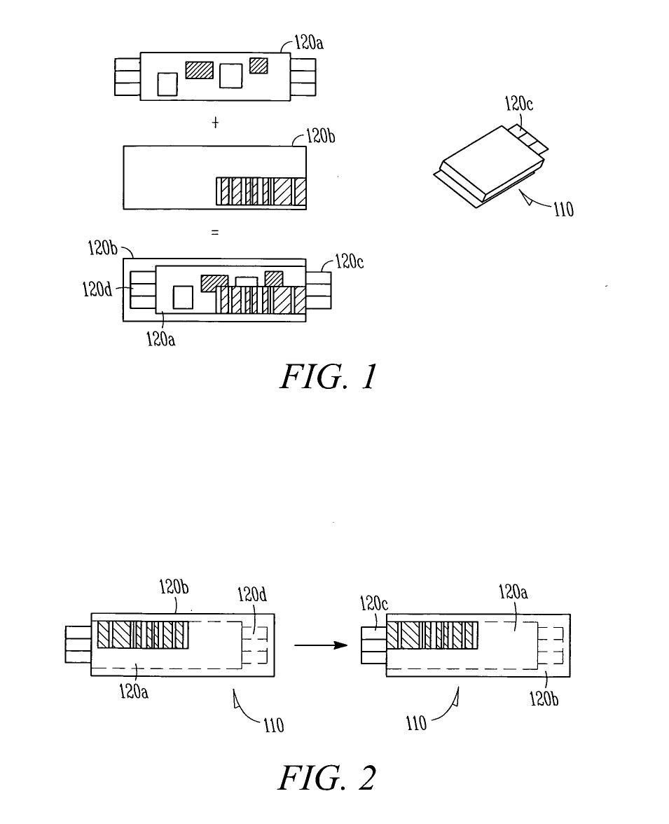

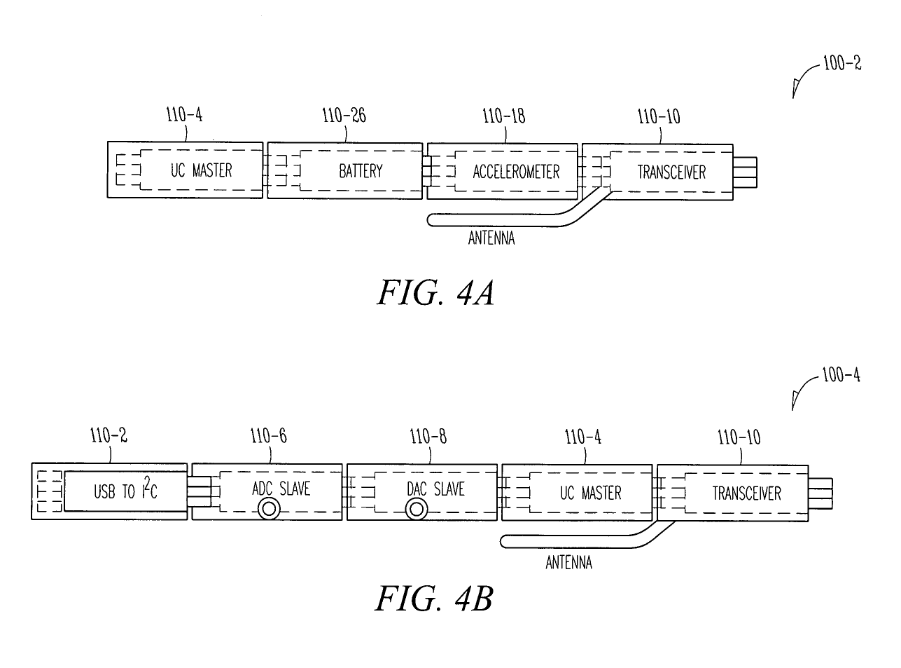

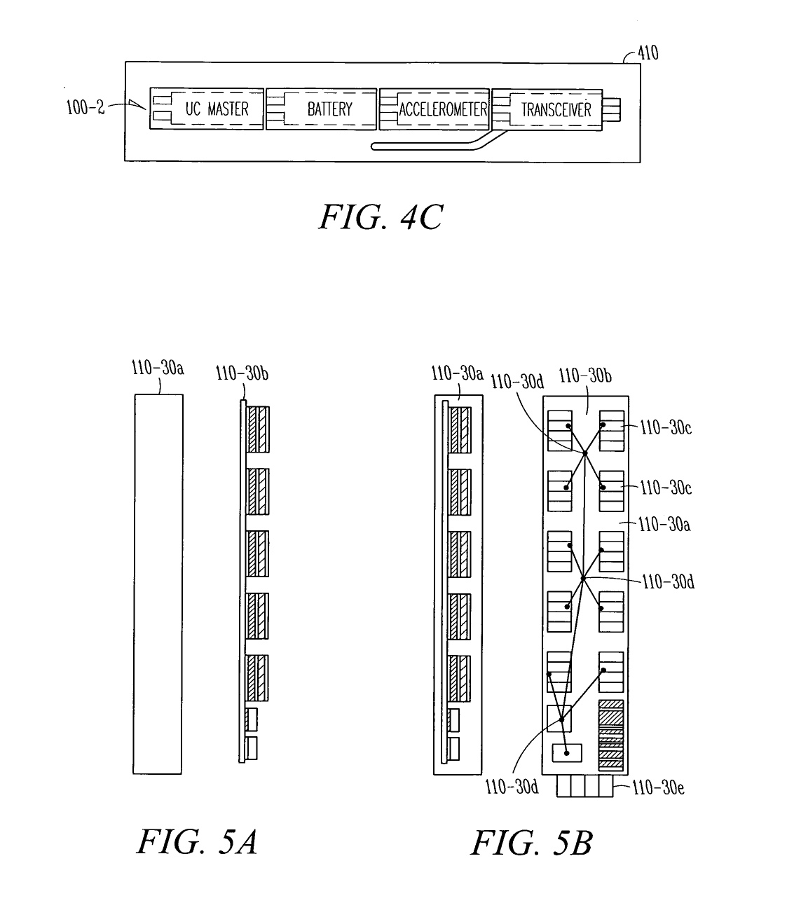

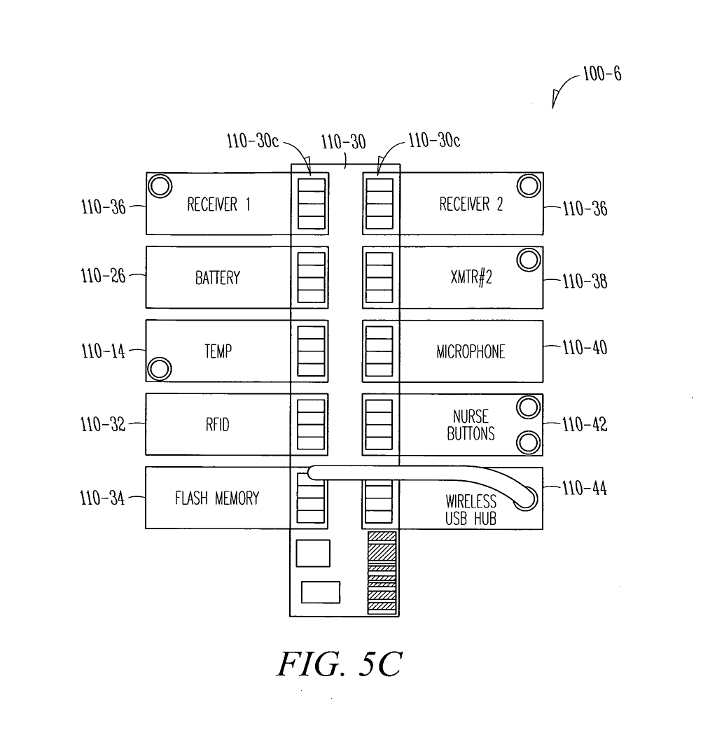

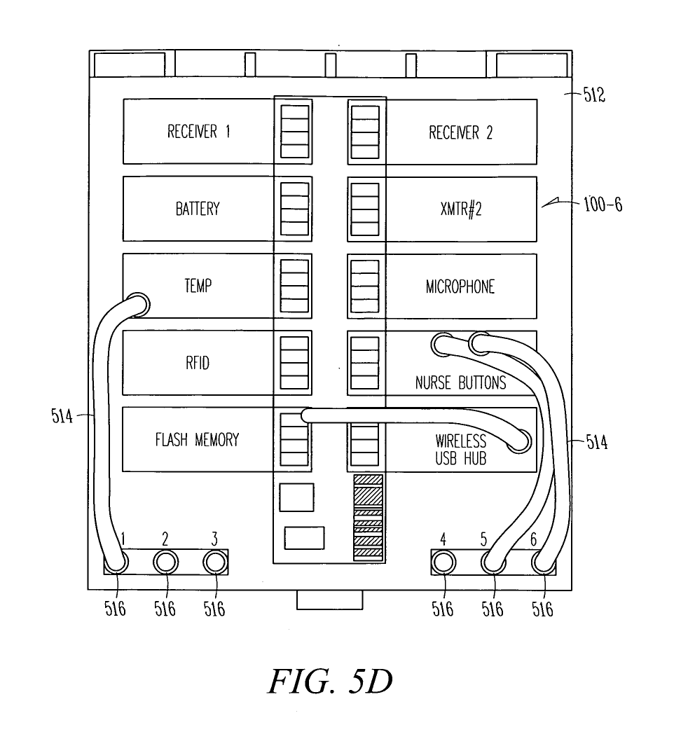

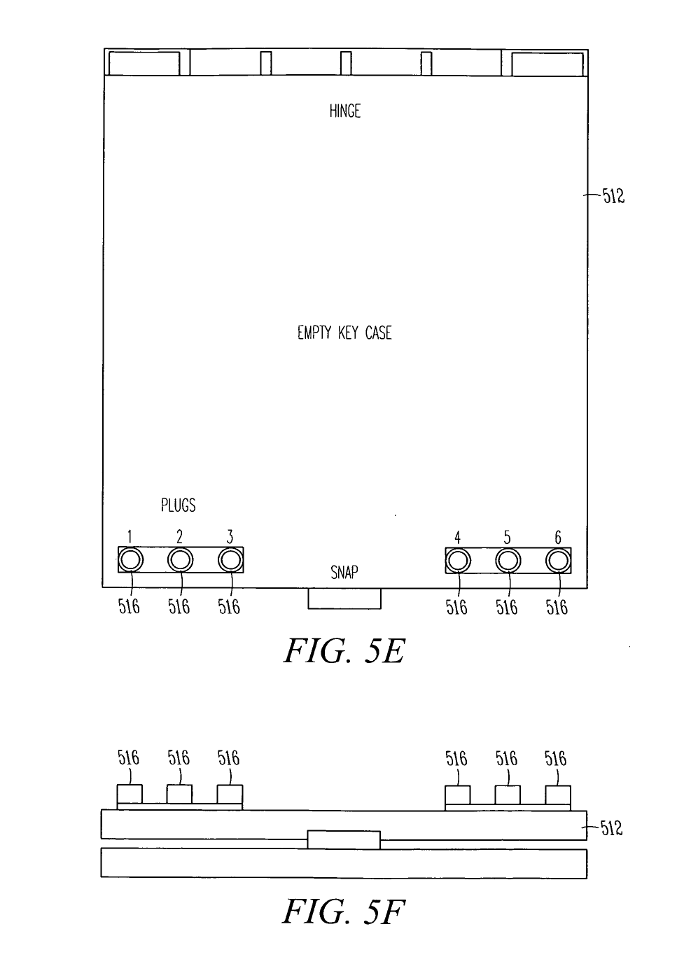

Referring now to FIG. 1, there is illustrated one example construction of a module 110 used to assemble a modular medical device 100 as illustrated for example in FIGS. 4A, 4B, 5A, 5B, 5C, 5D, 5E and 5F. Module 110 may include a circuit 120a mounted in a housing 120b, and male and female contacts 120c and 120d respectively, to connect the module to another module or bus. In one embodiment, module 110 includes a male connector 120e and a female receptacle 120f to receive a male connector 120e and to facilitate connection of the respective contacts 120b and 120c. Accordingly, in one embodiment as illustrated in FIG. 2, the modules 110 may be plugged together end to end. The module housing 110g may include a bar code 110h to enable identification using a bar code reader. According to another embodiment, the modules 110 are mechanically and electrically compatible to allow them to be assembled together in large variety of combinations to provide desired functionality. Modules 100, when assembled into a modular medical device 100, work together as a unit to provide one or more dedicated medical functions or systems. In one embodiment, modules 110 are constructed so as to share mechanical and electrical interfaces suited to the medical environment. Functionally speaking, many modular medical device 100 embodiments may be miniaturized, highly modular microcomputer platforms with standard common buses permitting the mixing and matching of modules 110. According to another embodiment, modules 110 are engineered for the health care environment to be resistant to accumulation of bacteria or viruses, and to allow for sterilization,

According to one example embodiment, the following minimum selection of modules 110 may be provided to create modular devices 100 according to the inventive subject matter:
1. Data/memory module. This may be a flash drive like module with a controller (e.g., universal serial bus (USB) controller) and large flash memory.
2. Communications module. This might be a simple USB or wireless USB or a multi-master serial computer bus (termed “I²C” herein) socket for manual downloading or it can be a transmitter module transmitting at cell phone or other frequencies, again with a controller.
3. Controller module. This module orchestrates the behaviors of other modules. It can be simple or it can have microprocessor level capabilities. Again, it requires a controller for the communication bus it sits on.
4. Sensor module. This module will provide data to the data module as controlled by the controller module. It may also contain its own internal controls and, again, will require a bus controller to be able to communicate with other modules on the bus.
5. Battery module. This can be a low resistance-high current battery to power strong data transmissions over long distances or a rechargeable module intended for continuous use or any other power module specialized for a particular power profile. A degenerate instance of a power module might be a cord to a transformer brick.

Thus, according to one example embodiment, a modular medical device 100 may integrate various types of modules including health sensor modules, controller modules, wireless transmitter or transceiver modules, data storage modules, networking modules, and battery modules. Using these various modules 110, the method and apparatus of the inventive subject matter enables health professionals to “prescribe” medical devices 100 on a per-patient basis, and each such system can be straightforwardly constructed, at a nurse's station or pharmacy. Modular devices 100 may be assembled, for example, by hot plugging prescribed modules 110 together on a shared mechanical and electrical computer bus, forming a small, light, wearable, programmable, medical device. Such devices 100 may be used, for example, to monitor, log, and wirelessly report a patient's health parameters.

According to another example embodiment, the modules 110 may include more than one capability or function, and so are in no way limited to a single function or capability per module. For example, it is contemplated that many modules will include at least some storage or controller capability together with other functionality. Or, it is contemplated that certain combinations of functions and capabilities will be desired to have as “stock” modules to be used in combination with other modules of the same or differing capabilities.

A modular medical device 100 according to one embodiment logs incoming data from a sensor in a data storage or memory module under the control of a controller module, and downloads or transfers the data via its communication module. According to another example embodiment, the modular medical device 100 may include a function module capable of accepting program downloads that may be executed to provide software-driven functions, such as filtering health data acquired by a sensor or otherwise acquired by the medical device 100 and determine whether these data should trigger wireless alarms and data uploads or transfers to external devices.

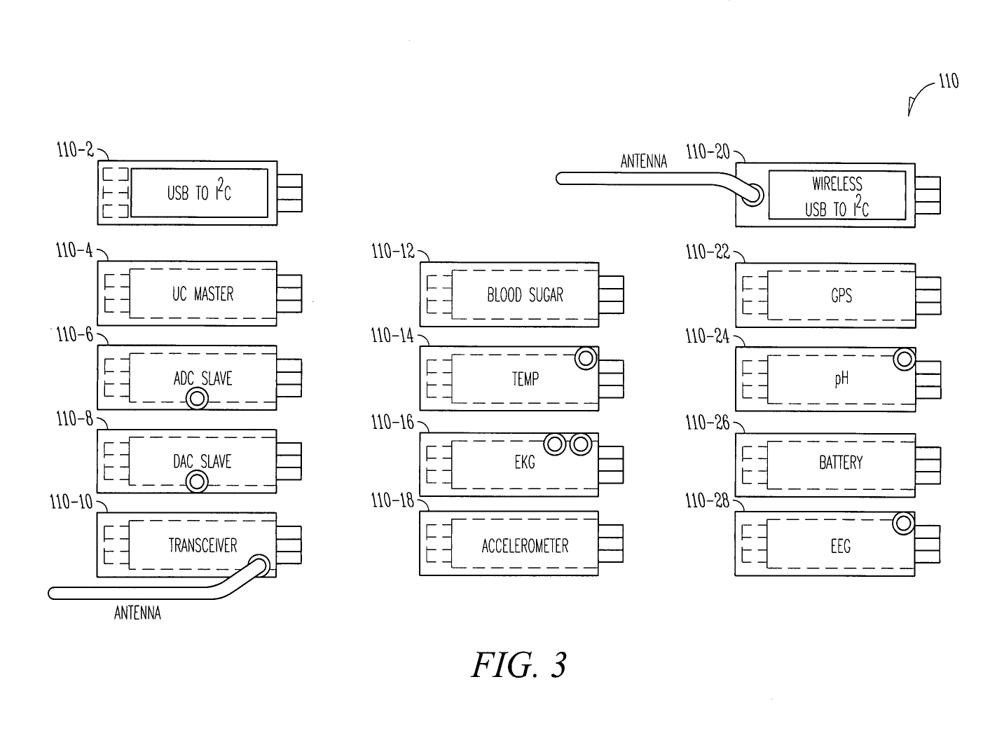

FIG. 3 illustrates a selection of modules 110 that may be combined in any desired configuration. The modules in FIG. 3 include a wired USB to I²C bidirectional translator module 110-2, a master microcontroller module 110-4, an analog to digital (A2D) module 110-6, a digital to analog (D2A) converter module 110-8, a transceiver module 110-10 having an antenna, a blood sugar monitor module 110-12, a temperature measurement module 110-14, an electrocardiogram module 110-16, an accelerometer module 110-18, wireless USB to I²C bidirectional translator module 110-20, a global positioning system (GPS) module 110-22, a pH measurement module 110-24, a battery module 110-26 and an electroencephalogram (EEG) module 110-28. Other modules not shown in FIG. 3 include receiver modules, microphone modules, radio frequency identification (RFID) modules, transmitter modules, blood pressure measurement modules, and USB hub modules.

Modules 110, in one embodiment, are color coded to help distinguish between and identify particular types of modules. Accordingly, modules of the same type or kind may be of the same color and have the same color coding. According to one embodiment, the modules 110 are all connected to a serial multimaster I²C bus with data, clock, power terminals (Vdd), and ground (GND) wires, wherein the bus is provided on each of the individual modules. The wired and wireless USB to I²C bidirectional translator modules 110-2 or 110-20 enable a USB wireless master node (for example in the nurses station) to control a modular medical device 100 containing such a module, or a enable wired USB connections to a master node to download data stored in a modular medical device 100 not containing a transmitter.

Referring to FIGS. 4A and 4B, there are illustrated two example embodiments 100-2 and 100-4 of (unpackaged) modular medical devices that may be formed from a linear concatenation of modules 110 by plugging the modules in a linear chain, each plugged into the next, connected for example by the I²C serial bus or other busses compatible therewith. Device 100-2 includes a microcontroller module 110-4, a battery module 110-26, an accelerometer module 110-18 and a transceiver module 110-10. Device 100-4 includes a USB to I²C bidirectional translator module 100-2, an A2D converter module 110-6, a D2A converter module 110-8, a microcontroller module 110-4 and a transceiver module 100-10. Device 100-2 may, for example, be used to transmit “fall down” alarms to a remote master node to indicate that a patient has fallen down. Device 100-4 may be used to attach to one or more instruments requiring A/D and D/A interfaces. FIG. 4C illustrates an example embodiment wherein the device 100-2 is enclosed in a housing 410. In the embodiment of FIG. 4C, housing 410 may be a cylindrical container, and the modules 110 may be sealed in the container using a material that is transparent to electromagnetic radiation, such as but not limited to Teflon.

Alternatively, as illustrated in FIGS. 5A, 5B and 5C, a modular medical device 100-6 according to the inventive subject matter may be formed from a “daisy chain” of modules 110, and connected to a USB hub module 110-30. FIGS. 5A and 5B illustrate a hub module package 110-30a and a hub module circuit board assembly 110-30b including female USB connectors 110-30c to receive and connect to male connectors on the respective USB modules 110. FIG. 5B illustrates a side and top view of an assembled hub module 110-30 illustrating hub bus circuits 110-30d including three levels of USB hierarchy. Module 110-30 may also include a male USB connector 110-30e to connect to one or more additional modules. For example USB module 110-30 may be connected to a microcontroller module through connector 110-30e, or possibly remotely (wirelessly) mastered. According to another embodiment, Type A USB connectors may be used for connecting USB-type modules to the hub module 110-30, or for other connections of the USB modules. These connectors are rugged and reliable and permit hot swapping of USB modules 110 into the hubs. Alternatively, Type B connectors are an option. USB connectors may provide two wires for power (for example 5V and GND) and a twisted pair to carry the data. According to one example embodiment, connectors for the modules described herein may be adapted to use magnetic force to hold the connectors together. According to another example embodiment, modules 110 include both male and female connectors to allow connection to more than one other module 110. Alternatively, modules 110 may have only one connector. According to another embodiment, an operating system on the microcontroller module includes one or more computer programs accessing module information stored on the module, in an on-board memory such as a flash module, or stored in a device registry available on a remote server, to recognize USB modules by type, function and/or communication protocols.

In the example embodiment of FIG. 5C, eight USB function modules 110 are plugged into the wireless USB module 110-30. This modular medical device 100-6 can run on battery power from module 110-26, has an RFID identification module 110-32, flash storage module 110-34, wireless receiver and transmitter modules 110-36 and 110-38, a microphone module 110-40, and a button module 110-42. Receiver and transmitter modules 110-36 and 110-38, temperature module 110-14 and button module 110-42 may include a shielded plug connector 510 for receiving a wire connector, for example a two or four wire connector. A wireless hub module 110-44 provides for wireless connectivity for the device 100-6. In one embodiment, this embodiment of system 100-6 may fit in a shirt pocket.

Referring to FIG. 5D, 5E and 5F, in which FIG. 5D illustrates a medical device 110-6 mounted in a packaged or container 512 wherein the modules 110 are sealed in the case. FIG. 5E and 5F illustrate top and end views of an empty case 512. Case 512 may also be transparent to electromagnetic radiation. If wires 514 are connected to one or more modules 110 mounted in the case, the wires can be routed from the modules 110 to the outside via plugs 516 that pass through the case while maintaining a seal.

In one example embodiment, the modules 110 described herein may be about one to two inches long, about one-half inch wide, and about one-quarter inch thick. However, there is no size requirement for the modules, and they may be smaller or larger in any dimension. In one embodiment, the modules 110 are flexible, “cheap,” reusable hardware sensing devices. In another embodiment, the modular medical devices formed from the modules can continually capture data from their environments and, based on some internal or external criteria, convey these historical data to an external device or system. In another embodiment, the modules 110 of a modular medical device as described herein may be permanently encased or “potted” so as to become a multi-module medical device.

Further, according to various embodiments, EKGs (heart), EEGs (brain), EOGs (eyes) can be monitored, recorded and transmitted by a modular medical device. Blood, urine and saliva chemistry may be sensed and forwarded to remote physicians or caregivers, using sensor modules for these functions. In one embodiment, certain combinations of modules 110 such as are described herein may become standard due to their frequent joint use in medical sensing. Standardization in one embodiment allows the management of both acute and chronic health disorders. Further, according to one example embodiment, the modular medical devices disclosed herein are used in hospitals to create wireless monitoring or data collection and reporting systems that do not need to be hard-wired to bed-side equipment. The modular medical devices can thus eliminate these spaghetti-like network of wires and sensors which is so encumbering (and expensive) for both patients and health professionals. In addition, as referred to above, the modules may be color coded and bar coded to aid in picking and assembling the correct modules.

In one example embodiment the modular medical devices may be ruggedized in such a way that the entire modular medical device structure remains mechanically and electrically well integrated. For example, USB “Type A” connections may be used. Connections between modules 110 may also be ruggedized by shrink wrapping the assembled modules. Alternatively, the modules may be potted together by placing the system in a mold, filling the mold with a potting material such as an epoxy-based compound, and letting the potting material set. Many other approaches to ruggedizing assembled modules 110 may be used.

Modules 110 for wireless communication may include short range low power options using infrared (IR), for example according to the Bluetooth standard or others, and may be used within hospital rooms or bedrooms. Mid-range wireless technologies, such as the WiFi standard as for example expressed in the 802.11b or 802.11g or successor or alternative standards, work for example for distances under about 110 meters. Longer range wireless technologies such as that provided by the WiMAX standard or equivalents or alternatives, may work over a few kilometers and might be useful for communicating over wider areas. Mobile phone data options are another alternative, and can communicate up to several miles to a cell site among a network of such cell sites. The tradeoff possibilities between power consumption, bandwidth, range, network connectivity, and other parameters are many can be adjusted in assembling a modular medical device by choosing a communication module suitable for the particular health care environment of each particular patient. Further, because privacy is an important issue in a health setting, encryption techniques such as using a virtual private network (VPN) and/or wired equivalent privacy (WEP) or Wi-Fi protected access (WPA) or other security protocol can be implemented in modular medical devices.

In another embodiment, a modular medical device may be formed using the modular approach described herein, from a transceiver module, a buzzer module, a global positioning system (GPS) module, a microphone module, an accelerometer module, a rechargeable battery module, and a flash memory module with enough memory to store data. Such a modular medical device 100 may be attached to a belt or other article of a subject's clothing, for example special needs child or elderly patient suffering cognitive loss, and generate buzzer reminders for the subject, send GPS coordinates to caregivers, send audio to caregivers, recognize falls and other impacts and transmit alarms to remote sites, and be dynamically and remotely programmed.

For geriatric care, an individualized modular medical device may be prescribed to a person whose balance was poor and frequently fell down, where blood sugar was an issue and where cardiac behavior needed monitoring. The associated sensor modules in this embodiment may be stacked into one modular medical device as prescribed by a doctor. In another embodiment, a pill monitor module with for example a liquid crystal display (LCD) indicator could be integrated into a modular medical device for the geriatric patient.

In other embodiments, modular medical devices according to the inventive subject matter may be used to monitor other medical instrumentation. For example, when bags of fluid fill to overflowing, an attached modular medical device may transmit a wireless “overflow alarm.” Conversely, a modular medical device may remind nurses when fluid reservoirs run dry. Patients may wish to have a push-button on a personal, ambulatory modular medical devices capable of forwarding requests to the nursing staff instead of the having the pushbutton tied to their bed as is current practice.

In other embodiments, doctors may use a modular medical device to enable particular remote diagnoses, selecting from a family of sensors and data communicator modules 110. Such specialized modular medical diagnostic devices are, in one embodiment, standardized for diagnosing different illnesses and health issues and “potted” to force permanent integration of the appropriate modules into a disease recognizing instrument for each distinguishable disease or health problem. A collection of several modular medical devices may also contain module firmware sufficient to act as an integrated network, enabling the sharing and forwarding of information to one another and thereby supporting reliability through redundant network connections.

Thus, the modular medical devices described herein provide for the integration of functionalities of “small” form factor modules 110 with common mechanical and electrical interfaces, permitting “mix and match” individualization of a “medical device” using such modules. For a given implementation, it may be as important what functionalities are not integrated into a modular medical device as what functionalities are included. Thus the inventive subject matter permits a level of low cost patient-centered optimization with great flexibility of configuration, and physical and electrical standardization, “miniaturization” (to human sizes), and modularization and mechanical “hardening” of connections sufficient to provide a “universal” sensor tool family optimized for health care needs.

According to another example embodiment, a method of the disclosed inventive subject matter provides for the assembly of medical devices 100 by medical personnel such as nurses, doctors or medical technicians, or by pharmacists or pharmacy personnel. Further, the systems 100 may be sold or rented or leased to patients. Systems 100 that are rented or leased may be returned to the renting or leasing entity, demodularized if desired, tested to ensure proper operation, and reused. Systems 100 may be distributed alternatively through retail stores, or over the Internet.

Thus, the modular medical devices described herein provide the ability to “prescribe” medical devices on a per-patient basis. Such prescriptions for example could be “filled” by medical personnel in the medical establishment, for example in a hospital, clinic or in a pharmacy. Such prescribed devices can be sold to patients, for example covered by insurance, or be rented or leased. Such prescribed medical devices might also be ordered over the Internet and sent directly to patients' homes. Accordingly, there is provided a convenient and optionally wireless “universal” medical tool that helps enable, among other things, the elderly to live at home rather than in nursing homes, and to potentially greatly reduce costs by modularizing health monitors, using, in one embodiment, standardized, re-usable, and miniaturized modules.

1. Apparatus comprising:
a medical device formed from a plurality of modules adapted to be directly plugged together or to a bus via connectors of the modules so that the modules can function together as a system;
wherein the modules are selected from a group of modules adapted to be used in combination to perform one or more medical functions for a particular patient;
wherein the plurality of modules forming the medical device includes at least one capability to store data, at least one capability to perform a communication function, at least one capability to control the medical device, at least one capability to provide a sense function, and at least one capability to provide energy to the medical device; and
further wherein the plurality of modules are mechanically and electrically compatible to allow them to be assembled together in a variety of combinations to provide desired functionality.

2. Apparatus according to claim 1 wherein the module with the capability to perform a communication function includes a mechanical connector or a wireless transmitter module to send information to a device outside the medical device.

3. Apparatus according to claim 1 wherein the module with the capability to control the medical device controls the behaviors of at least one of the other modules.

4. Apparatus according to claim 1 wherein the connected modules are shrink-wrapped or potted together to prevent them from being disassembled by the patient.

5. Apparatus according to claim 1 wherein the module with the capability to provide a sense function senses a physiological parameter.

6. Apparatus according to claim 1 wherein the module with the capability to provide a sense function senses a physical event.

7. A method comprising:
assembling a medical device from a plurality of modules, the assembling comprising directly plugging the modules together or to a bus via connectors of the modules so that the modules can function together as a system and selecting the modules forming the medical device based on one or more medical functions to be performed by the medical device for a particular patient;
wherein the modules are selected from a group of modules adapted to be used in combination to perform one or more medical functions;
wherein the plurality of modules forming the medical device includes at least one capability to store data, at least one capability to perform a communication function, at least one capability to control the medical device, at least one capability to sense a physiological parameter, and at least one capability to provide energy to the medical device; and
further wherein the plurality of modules are mechanically and electrically compatible to allow them to be assembled together in a variety of combinations to provide desired functionality.

8. A method according to claim 7 wherein the modules are connected through a bus mounted in a housing such that the modules are plugged into the bus and are positioned in the housing.

9. A method according to claim 7 wherein the medical device is prescribed for the patient by a pharmacist, physician or other medical personnel.

10. A method according to claim 7 wherein the module with the capability to perform a communication function includes a mechanical connector or a wireless transmitter module to send information to a device outside the medical device.

11. A method according to claim 7 wherein the module with the capability to control the medical device controls the behaviors of at least one of the other modules.

12. A method according to claim 7 wherein the connected modules are shrink-wrapped or potted together to prevent them from being disassembled by the patient.

13. A method according to claim 7 wherein the module with the capability to provide a sense function senses a physiological parameter.

14. A method according to claim 7 wherein the module with the capability to provide a sense function senses a physical event.

15. A method according to claim 7 wherein the medical device is rented to a patient and returned by the patient and disassembled after use.
